(19) United States
(12) Patent Application Publication
(10) Pub. No.: US 2011/0275841 A1
(43) Pub. Date: Nov. 10, 2011

(54) PREPARATION OF DOCETAXEL

(75) Inventors: Rajasekhar Kadaboina, Hyderabad (IN); Sekhar Munaswamy Nariyam, Hyderabad (IN); Lankeshwara Rao Matti, Hyderabad (IN); Sridhar Vasam, Warangal (IN); Siva Reddy Makireddy, Guntur (IN); Shravan Kumar Komati, Nalgonda (IN)

(73) Assignees: DR. REDDY'S LABORATORIES, INC., Bridgewater, NJ (US); DR. REDDY'S LABORATORIES LTD., Hyderabad (IN)

(21) Appl. No.: 13/112,165

(22) Filed: May 20, 2011

Related U.S. Application Data
(63) Continuation of application No. PCT/US2009/065283, filed on Nov. 20, 2009.
(60) Provisional application No. 61/148,219, filed on Jan. 29, 2009.

(30) Foreign Application Priority Data
Nov. 20, 2008 (IN) 2884/CHE/2008

(51) Int. Cl. C07D 305/14

(52) U.S. Cl. 549/511; 549/510

(57) ABSTRACT

The present invention relates to docetaxel and processes for preparing docetaxel, including process-related intermediates. The present invention also relates to processes for preparing substantially pure docetaxel and intermediates.

### INTRODUCTION

Aspects of the present invention relate to docetaxel and processes for preparing docetaxel, including process-related intermediates. Aspects of the present invention also relate to processes for preparing substantially pure docetaxel and its intermediates.

Docetaxel, an antineoplastic agent belonging to the taxoid family, has a chemical name (2R,3S)-N-carboxy-3-phenylisoserine, N-tert-butyl ester, 13-ester with 5β-20-epoxy-1,2α,4,7β,10β,13α-hexahydroxytax-11-en-9-one 4-acetate 2-benzoate, and in its anhydrous form can be represented by structural Formula I.

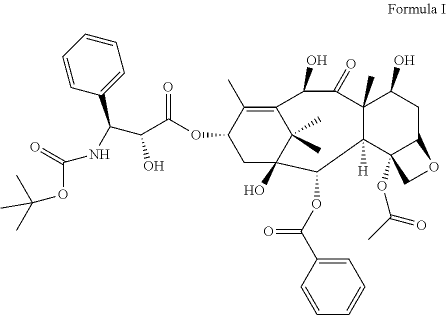

Docetaxel is useful in the treatment of the malignant neoplasms, such as solid tumors, and other malignancies.

U.S. Pat. No. 4,814,470 discloses docetaxel, its stereoisomeric forms, pharmaceutical compositions containing docetaxel, and their use in the treatment of acute leukemia and solid tumours.

U.S. Pat. No. 5,476,954 discloses a process for preparing docetaxel that involves:

(a) reacting 4-acetoxy-2α-benzoyloxy-5β,20-epoxy-1,13α-dihydroxy-9-oxo-7β,10β-bis[(2,2,2-trichloroethoxy)carbonyloxy]-11-taxene with (4S,5R)-3-tert-butoxycarbonyl-2,2-dimethyl-4-phenyl-5-oxazolidinecarboxylic acid in the presence of N,N′-dicyclohexyl carbodiimide (DCC), 4-(N,N′-dimethylamino)pyridine (DMAP), and toluene to give 4-acetoxy-2α-benzoyloxy-5β,20-epoxy-1-hydroxy-9-oxo-7β,10β-bis[(2,2,2-trichloroethoxy)carbonyloxy]-11-taxen-13α-yl(4S,5R)-3-tert-butoxycarbonyl-2,2-dimethyl-4-phenyl-5-oxazolidinecarboxylate, purifying the product by flash chromatography using dichloromethane/methanol as eluent;

(b) treating the compound obtained in step (a) with formic acid to give 4-acetoxy-2α-benzoyloxy-5β,20-epoxy-1-hydroxy-9-oxo-7β,10β-bis[(2,2,2-trichloroethoxy)carbonyloxy]-11-taxen-13α-yl(2R,3S)-3-amino-2-hydroxy-3-phenyl propionate, and purifying the product by flash chromatography using dichloromethane/methanol as eluent;

(c) converting the compound obtained from step (b) to 4-acetoxy-2α-benzoyloxy-5β,20-epoxy-1-hydroxy-9-oxo-7β,10β-bis[(2,2,2-trichloroethoxy)carbonyloxy]-11-taxen-13α-yl(2R,3S)-3-tert-butoxycarbonylamino-2-hydroxy-3-phenylpropionate, by the action of di-tert-butyl dicarbonate in the presence of an inorganic or organic base; and

(d) converting the compound obtained in step (c) to docetaxel by deprotection of the hydroxyl protecting group, 2,2,2-trichloroethoxycarbonyl, from the 7β and 10β positions using zinc in the presence of acetic acid and methanol.

The present inventors have observed that carrying out step (b) as disclosed in U.S. Pat. No. 5,476,954 (“the '954 process”) requires a reaction time of about 4.5 hours, and using excess molar equivalents of di-tert-butyl dicarbonate (about 16 equivalents) in step (c) as required in the '954 process leads to the formation of increased amounts (up to 5%) of each of the related impurities I and II and formation of related impurity III in amounts more than 5%. The structures of the three principal impurities formed in the '954 process are shown in Table 1.

TABLE 1

| Impurity | Structure |
|---|---|
| Impurity I | 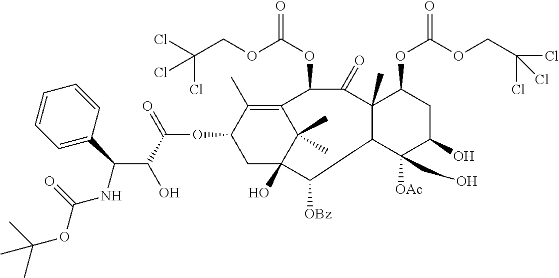 |
| Impurity II | 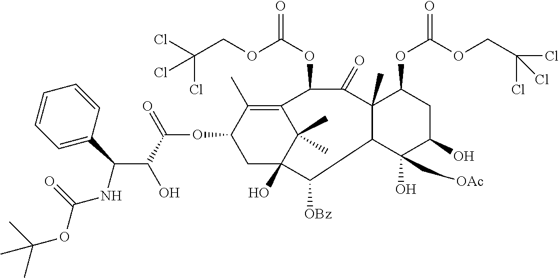 |
| Impurity III | 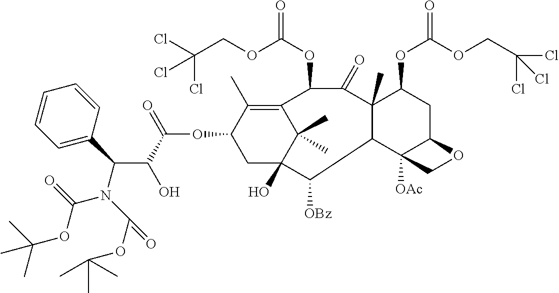 |

The present inventors have also observed that use of a molar excess of acetic acid in combination with the solvent, such as methanol in step (d) of the '954 process, results in the formation of increased levels of the impurities shown in Table 2.

TABLE 2

| Impurity | Structure |
|---|---|
| Impurity V | 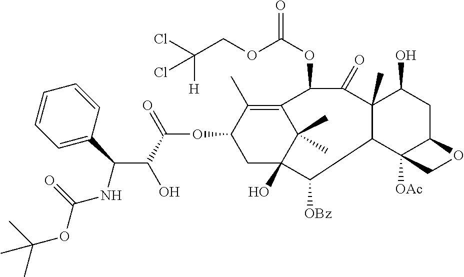 |
| Impurity VI | 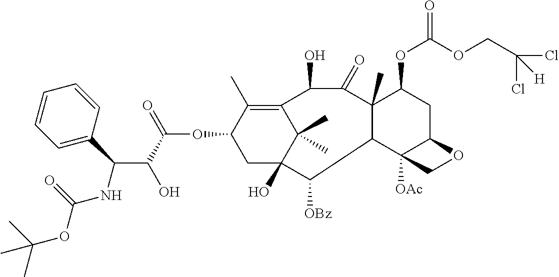 |
| Impurity VII | 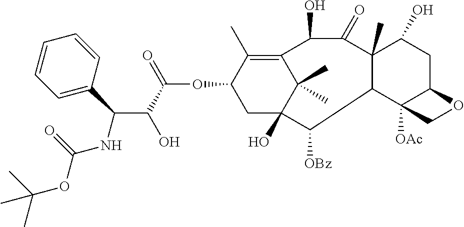 |

U.S. Pat. No. 5,686,623 discloses a process for preparing 4-acetoxy-2α-benzoyloxy-5β,20-epoxy-1-hydroxy-9-oxo-7β,10β-bis(2,2,2-trichloroethoxycarbonyloxy)-tax-11-en-13-α-yl(4S,5R)-3-tert-(butoxycarbonyl)-2,2-dimethyl-4-phenyl-1,3-oxazolidine-5-carboxylate, by reacting 4-acetoxy-2α-benzoyloxy-5β,20-epoxy-1,13α-dihydroxy-9-oxo-7β,10β-bis[(2,2,2-trichloroethoxy)carbonyloxy]-11-taxene with (4S,5S)-3-tert-butoxycarbonyl-2,2-dimethyl-4-phenyl-1,3-oxazolidine-5-carboxylic acid in the presence of DCC, DMAP and anhydrous toluene at 72° C. for 16 hours, followed by purification using preparative silica thin layer chromatography, eluting with an ether/hexane/dichloromethane (5:20:75 by volume) mixture, which is further converted to docetaxel by a process similar to that disclosed in US '954 patent.

U.S. Pat. No. 6,197,980 discloses a process for preparing docetaxel by reacting 4-acetoxy-2α-benzoyloxy-5β,20-epoxy-1β-hydroxy-7β,10β-bis[(2,2,2-trichloroethoxy)carbonyloxy]-9-oxo-11-taxen-13α-yl (2R,3S)-3-t-butoxycarbonylamino-3-phenyl-2-hydroxy propionate with zinc and acetic acid in the presence acetonitrile.

U.S. Pat. No. 5,616,739 discloses a process for preparing 4-acetoxy-2α-benzoyloxy-5β,20-epoxy-1β,7α,10α-trihydroxy-9-oxo-11-taxen-13α-yl (2R,3S)-3-amino-2-hydroxy-3-phenylpropionate by reacting 4-acetoxy-2α-benzoyloxy-5β,20-epoxy-1β-hydroxy-7β,10β-bis[(2,2,2-trichloroethoxy)carbonyloxy]-9-oxo-11-taxen-13α-yl (4S,5R)-3-(2,2,2-trichloroethoxycabonyl)2,2-dimethyl-4-phenyl-5-oxazolidine carboxylate with zinc and glacial acetic acid in the presence of ethyl acetate.

The reaction conditions as disclosed in various disclosures for preparing docetaxel and its intermediates may result in the generation of increased level of process related impurities. These impurities may be, for example, starting materials, by-products of the reaction, products of side reactions, or degradation products. The removal of these impurities at each stage of the process using expensive purification techniques, such as flash chromatography or column purification in multiple stages may result in lower yield, which is not suitable for an industrial-scale process.

Therefore, there is an ongoing need for improved processes for the preparation of docetaxel with high purity and yield, which processes are eco-friendly, cost-effective, and well suited for an industrial scale.

### SUMMARY

The present invention relates to docetaxel and processes for preparing docetaxel, including process-related intermediates. The present invention also related to processes for preparing substantially pure docetaxel and its intermediates thereof.

The present invention provides process for preparing docetaxel, which process comprises:

a) reacting 4-acetoxy-2α-benzoyloxy-5β,20-epoxy-1-hydroxy-9-oxo-7β,10β-bis[(2,2,2-tri-chloroethoxy)carbonyloxy]-11-taxen-13α-yl (4S,5R)-3-tert-butoxycarbonyl-2,2-dimethyl-4-phenyl-5-oxazolidinecarboxylate of formula II,

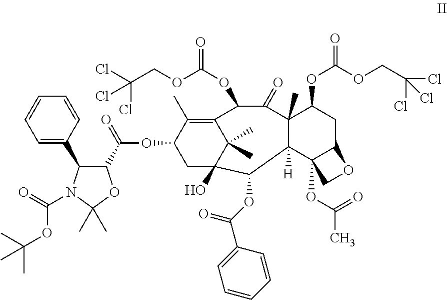

with formic acid at temperatures about 20 to 40° C. for less than 2 hours, to obtain 4-acetoxy-2α-benzoyloxy-5β,20-epoxy-1-hydroxy-9-oxo-7β,10β-bis[(2,2,2-trichloro ethoxy)carbonyloxy]-11-taxen-13α-yl (2R,3S)-3-amino-2-hydroxy-3-phenylpropionate intermediate compound of formula III;

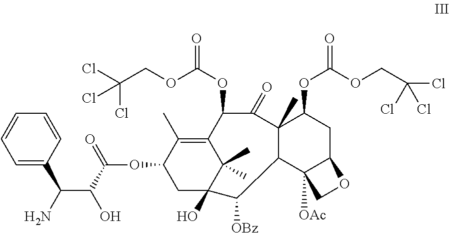

b) reacting the compound of formula III with 1 to 1.5 molar equivalents of di-tert-butyl dicarbonate in a bi-phasic medium to obtain 4-acetoxy-2α-benzoyloxy-5β,20-epoxy-1-hydroxy-9-oxo-7β,10β-bis[(2,2,2-trichloroethoxy)carbonyloxy]-11-taxen-13α-yl(2R,3S)-3-tert-butoxycarbonyl amino-2-hydroxy-3-phenylpropionate intermediate compound of formula IV; and

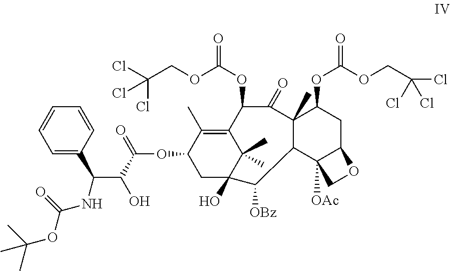

c) converting the compound of formula IV to docetaxel.

The present invention provides process for purifying the intermediate compound of Formula IV, which process comprises:

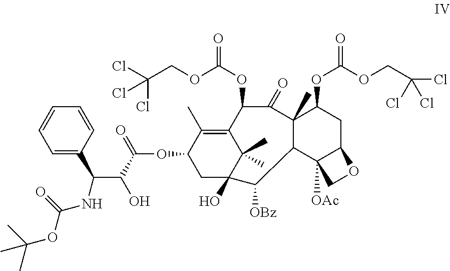

a) providing a solution of the compound of formula IV in an organic solvent;

b) combining the solution of step a) with an anti-solvent;

c) isolating the compound of formula IV; and

d) optionally repeating steps (a)-(c) to obtain a desired purity.

The present invention further provides processes for preparing docetaxel, an embodiment comprising reacting an intermediate compound of formula IV,

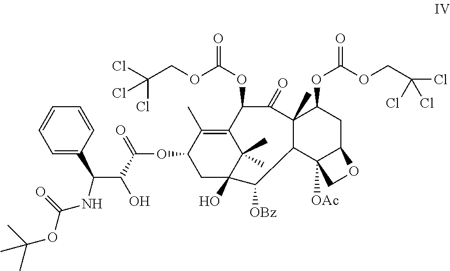

with zinc in the presence of an organic acid and a ketone solvent to give docetaxel.

The present invention further provides processes for preparing substantially pure docetaxel, embodiments comprising any of I, II, and II, or combinations thereof:

I. Purification Using a Nitrile and Ether:

a) dissolving the docetaxel in a solvent comprising a nitrile;

b) combining the solution of a) with an ether; and

c) isolating crystalline docetaxel.

II. Purification Using a Ketone, Halogenated Hydrocarbon, Ester, or any Mixtures Thereof; and Toluene:

a) combining docetaxel with a ketone, halogenated hydrocarbon, ester, or any mixtures thereof;

b) adding toluene to the solution or suspension; and

c) isolating crystalline docetaxel.

III. Purification Using a Ketone and an Ether:

a) dissolving docetaxel in a ketone;

b) adding ether to the solution; and

c) isolating crystalline docetaxel.

### BRIEF DESCRIPTION OF THE DRAWING

FIG. 1 illustrates an X-ray powder diffraction (XRPD) pattern of docetaxel, as prepared in Example 11.

### DETAILED DESCRIPTION

Percentage values described herein are expressed on a weight basis, unless the context indicates otherwise.

In an aspect, the present invention provides processes for preparing docetaxel, embodiments comprising:

a) reacting 4-acetoxy-2α-benzoyloxy-5β,20-epoxy-1-hydroxy-9-oxo-7β,10β-bis[(2,2,2-tri-chloroethoxy)carbonyloxy]-11-taxen-13α-yl (4S,5R)-3-tert-butoxycarbonyl-2,2-dimethyl-4-phenyl-5-oxazolidinecarboxylate of formula II,

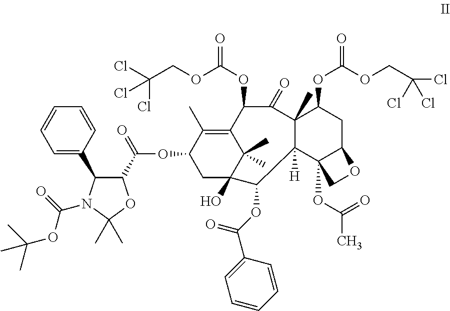

with formic acid at temperatures about 20 to 40° C. for less than 2 hours, to obtain 4-acetoxy-2α-benzoyloxy-5β,20-epoxy-1-hydroxy-9-oxo-7β,10β-bis[(2,2,2-trichloro ethoxy)carbonyloxy]-11-taxen-13α-yl (2R,3S)-3-amino-2-hydroxy-3-phenylpropionate intermediate compound of formula III;

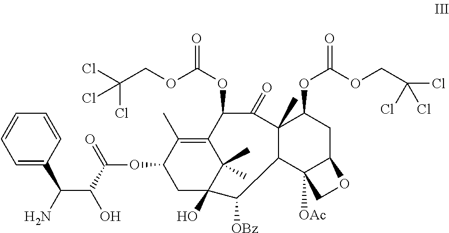

b) reacting the compound of formula III with 1 to 1.5 molar equivalents of di-tert-butyldicarbonate in a bi-phasic medium to obtain 4-acetoxy-2α-benzoyloxy-5β,20-epoxy-1-hydroxy-9-oxo-7β,10β-bis[(2,2,2-trichloroethoxy)carbonyloxy]-11-taxen-13α-yl(2R,3S)-3-tert-butoxycarbonylamino-2-hydroxy-3-phenylpropionate intermediate compound of formula IV; and

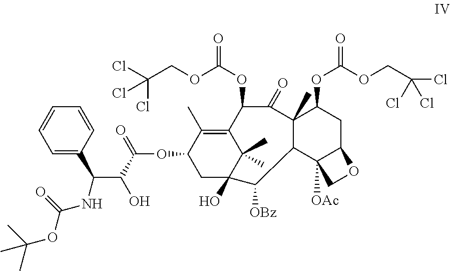

c) converting the compound of formula IV to docetaxel.

Step (a) involves the reaction of the compound of formula II with formic acid at temperatures about 20 to 40° C. for less than 2 hours.

The amount of formic acid used in step (a) is about 5-30 mL, per gram of the compound of Formula II. The process of step (a) may be carried out at temperatures about 20-40° C.

The present inventors found that conducting step (a) using 5-30 mL of formic acid, per gram of the compound of formula II, and at temperatures about 20-40° C. will not only facilitate completion of the reaction in less than 2 hours, but also reduce the formation of related impurities, particularly the principal impurities, i.e., impurities I and II, to amounts less than about 1%. In embodiments, the process results in amounts of these principal impurities less than about 0.5%.

After completion of the reaction, the reaction mass may be diluted with an organic solvent and optionally with water and/or brine solution, and stirred for a suitable time. For example, the organic solvents used include, but are not limited to: esters such as for example ethyl acetate, butyl acetate, isopropyl acetate and the like; ketones such as methyl isobutyl ketone and the like; ethers such methyl tert-butyl ether, dimethoxyethane, diethoxyethane and the like; and any combinations thereof. The organic layer may be separated and may optionally be washed with water and saturated bicarbonate solution. The process may be repeated if desired. The obtained reaction mass may optionally be concentrated before proceeding to the next reaction step.

Step (b) involves reacting 4-acetoxy-2α-benzoyloxy-5β,20-epoxy-1-hydroxy-9-oxo-7β,10β-bis[(2,2,2-trichloroethoxy)carbonyloxy]-11-taxen-13α-yl(2R,3S)-3-amino-2-hydroxy-3-phenylpropionate (i.e., the reaction product of step (a)) with 1 to 1.5 molar equivalents of di-tert-butyl dicarbonate. In an embodiment, the quantity of di-tert-butyl dicarbonate in step (b) may be about 1.2 molar equivalents, per molar equivalent of 4-acetoxy-2α-benzoyloxy-5β,20-epoxy-1-hydroxy-9-oxo-7β,10β-bis[(2,2,2-trichloroethoxy)carbonyloxy]-11-taxen-13α-yl (2R,3S)-3-amino-2-hydroxy-3-phenylpropionate. The reaction of step (b) may be achieved in a bi-phasic medium. The bi-phasic medium for step (b) may be obtained by charging water to the reaction mass or organic layer obtained in step (a).

The pH of the resulting reaction mass is adjusted using a base, e.g., sodium bicarbonate, either as a solid or its saturated solution or with an organic base for e.g., triethylamine, diispropylethylamine and the like. The pH of the reaction mass may be adjusted to a range of about 7-9. In embodiments, the pH of the reaction mass may be adjusted to about 7.5-8.5 using solid sodium bicarbonate.

Step (b) may be carried out at temperatures about 20-40° C. In embodiments, step (b) may be carried out at temperatures about 25-30° C.

After completion of the reaction, the organic layer may be separated and optionally washed with water. The organic layer obtained may be concentrated to about 3 mL per gram of the compound of formula II used, under high vacuum and at temperatures about 40-50° C.

The compound of formula IV may be precipitated by cooling the reaction mass to 25-30° C. and by adding a suitable anti-solvent, such as but not limited to alkanes, including, for example, n-heptane, n-hexane, cyclohexane, and the like.

The obtained solid may be separated using conventional techniques known in the art. After separation, the solid may optionally be washed, e.g., with an anti-solvent, and may optionally be further dried by techniques known in the art. The drying may be carried out at temperatures of about 35-70° C., optionally under reduced pressure, for any time periods necessary for obtaining a desired purity.

At the completion of step (b), the compound of formula IV contains the related impurity (Impurity III) in amounts less than about 1%.

Step (c) involves converting the compound of formula IV to docetaxel. The compound of formula IV obtained in step (b) may be converted to docetaxel using a process known in the art or by the process described hereinbelow.

Aspects of the present invention provides processes for purifying the intermediate compound of formula IV, embodiments comprising:

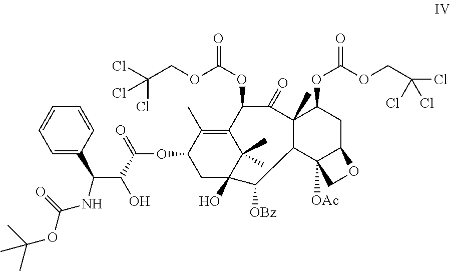

a) providing a solution of the compound of formula IV in an organic solvent;

b) combining the solution with an anti-solvent;

c) isolating the compound of formula IV; and

d) optionally repeating steps (a)-(c) to obtain a desired purity.

Purification step (a) involves providing a solution of the compound of formula IV in an organic solvent.

The organic solvents which may be used for providing a solution of the compound of formula IV include, but are not limited to: esters such as for example ethyl acetate, butyl acetate, isopropyl acetate and the like; nitriles such as acetonitrile, propionitrile and the like; halogenated alkanes such as dichloromethane, chloroform and the like; and any combinations thereof. In embodiments, the organic solvent may comprise acetonitrile.

The concentration of the compound of formula IV in the organic solvent may generally range from about 0.01 to about 1 g/mL. However the concentration of the compound of formula IV in the organic solvent selected is not critical, although the quantity of solvent employed is usually kept to a minimum to avoid excessive product losses during the crystallization of the solute (i.e., the compound of formula IV).

The solution of the compound of formula IV in the organic solvent may be prepared at temperatures ranging from about 25-75° C. Depending on the quantity of solvent used, the compound of formula IV may dissolve at a temperature of about 25-35° C., or the solution may need to be heated to elevated temperatures.

Purification step (b) involves combining the solution of step (a) with an anti-solvent.

Useful anti-solvents include, but are not limited to: ethers, such as, for example, diethyl ether, diisopropyl ether, methyl tert-butyl ether, and the like; and any combinations thereof. In embodiments, the anti-solvent may comprise diisopropyl ether.

Suitable temperatures for purification step (b) may range from about 0-50° C., or from about 25-35° C.

Purification step (c) involves isolating the compound of formula IV.

The solid formed after purification step (b) may be separated using conventional techniques known in the art. One skilled in the art may appreciate that there are many ways to separate a solid from the mixture. For example, it may be separated by filtration by gravity or suction, centrifugation, decantation, and the like. After separation, the solid may optionally be washed, e.g., with a suitable anti-solvent. In embodiments, the solid obtained is washed with petroleum ether.

The isolated solid may optionally be further dried by using conventional techniques. The drying may be carried out at temperatures of about 35-70° C., optionally under reduced pressure. The drying may be carried out for any time periods necessary for obtaining a desired purity, such as about 1 to about 25 hours, or longer.

After purification step (c), the compound of formula IV typically has a purity greater than or equal to about 92%, or greater than or equal to about 98%, and contains less than about 0.5% of each of the impurities I, II, and III, as determined using high performance liquid chromatography (HPLC).

Purification step (d) involves optionally repeating purification steps (a) through (c) to obtain a desired purity. Purification steps (a) through (c) may optionally be repeated to get a substantially pure compound of formula IV having purity greater than or equal to about 95%, or greater than or equal to about 98%, as determined using HPLC.

The present invention includes the compound of formula IV substantially free of its isomeric or related impurities. The term “substantially free of its isomeric or the related impurities” as used herein shall be understood to mean comprising isomeric or related impurities in an amount less than about 2%, or less than about 0.5%, or less than about 0.15%, or less than about 0.05%, of the identified compound.

Aspects of the present invention further provide processes for preparing docetaxel, embodiments comprising reacting an intermediate compound of formula IV,

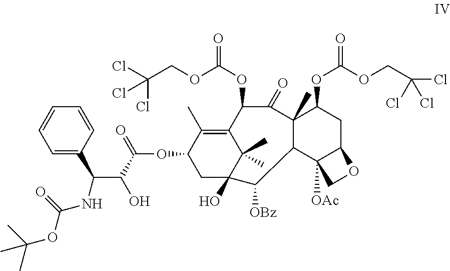

with zinc in the presence of an organic acid and a ketone solvent, to give docetaxel.

The amount of zinc utilized in step (a) may be about 1-30 molar equivalents, per mole of the compound of formula IV. In embodiments, about 16 moles of zinc, per mole of the compound of formula IV, may be used.

The organic acid used in the reaction may comprise acetic acid, propionic acid, butanoic acid, oxalic acid, and the like. In embodiments, acetic acid is used. The amount of the organic acid used in the reaction may range from about 1-5 mL, per gram of the compound of formula IV. In embodiments, about 1 mL of acetic acid, per gram of the compound of formula IV, may be used.

The ketone solvents that may be used in the process of step (a) include, but are not limited to, acetone, methyl isobutyl ketone, ethyl methyl ketone, and the like. In embodiments, acetone is used. The amount of ketone solvent used in the reaction may range from about 1-40 mL, per gram of the compound of formula IV. In embodiments, about 20 mL, or about 30 mL, of acetone, per gram of the compound of formula IV, may be used.

The process may be carried out at temperatures of about 25° C. to about the reflux temperature of the solvent used. In embodiments, it may be carried out at temperatures about 30-50° C.

It has been found that reacting the compound of formula IV with zinc in the presence of an organic acid and a ketone solvent gives docetaxel with increased purity, as compared to the prior processes that result in docetaxel having a content of impurities V and VI greater than about 5-6%.

Without wishing to be bound by a particular theory, it is believed that use of a smaller quantity of the organic acid and ketone solvent not only facilitates the reaction completion, but also decreases the formation of impurities such as impurities V, VI, and VII to less than about 2%, as determined using HPLC.

After completion of the reaction, the reaction mass may optionally be filtered through media such as a bed of diatomaceous earth, and washed with the ketone solvent.

The obtained filtrate may optionally be concentrated to a minimum volume, for example, 1-5 mL per gram of the compound of formula IV, and used in the next level of purification or extracted into another organic solvent, and/or may be isolated by precipitation to give docetaxel.

Docetaxel may be precipitated from the solution by addition of an anti-solvent. Useful anti-solvents include, but are not limited to: alkanes, such as, for example, n-heptane, n-hexane, cyclohexane, and the like; ethers, such as, for example, diethyl ether, diisopropyl ether, tetrahydrofuran (THF), 1,4-dioxane, dimethoxyethane, methyl tert-butyl ether, and the like; water; and any combinations thereof. In embodiments, water is used as an anti-solvent.

Suitable temperatures for solid crystallization may range from about 0° C. to about 50° C., or from about 25° C. to about 35° C.

The obtained solid may be separated using conventional techniques known in the art. After separation, the solid may optionally be washed with an anti-solvent.

The isolated solid may optionally be further dried by techniques known in the art. The drying may be carried out at temperatures of about 35° C. to about 70° C., optionally under reduced pressure, for any time periods necessary for obtaining a desired purity.

Docetaxel can be obtained from the present invention in a high yield of more than about 95%, with a high purity of more than about 90%.

The docetaxel obtained may optionally be further purified according to a process known in the art, or by column chromatography, or by crystallization processes, as per the processes disclosed in the present application.

Aspects of the present invention further provides processes for preparing substantially pure docetaxel, embodiments comprising one or more of I, II, and III.

I. Purification Using a Nitrile and Ether:

a) dissolving the docetaxel in a solvent comprising a nitrile;

b) combining the solution of a) with an ether; and

c) isolating crystalline docetaxel.

II. Purification Using a Ketone, Halogenated Hydrocarbon, Ester, or any Mixtures Thereof; and Toluene:

a) combining docetaxel with a ketone, halogenated hydrocarbon, ester, or any mixtures thereof;

b) adding toluene to the solution or suspension; and

c) isolating crystalline docetaxel.

III. Purification Using a Ketone and an Ether:

a) dissolving docetaxel in a ketone;

b) adding ether to the solution; and

c) isolating crystalline docetaxel.

In embodiments, the procedures of I, II, and III are performed in the recited sequence.

These steps for preparing substantially pure docetaxel are individually described below.

I. Purification in a Solvent Comprising a Nitrile and Ether.

a) Dissolving Docetaxel in a Solvent Comprising Nitrile.

Docetaxel obtained from any synthetic methods can be used as a starting material. Docetaxel (such as having purity about 85% or less) can be purified by crystallization of the present invention.

Docetaxel is dissolved in a solvent comprising a nitrile solvent, such as acetonitrile, propionitrile, and the like. In embodiments, docetaxel is dissolved in a solvent mixture of acetonitrile and dichloromethane.

Depending on the quantity of solvent used, docetaxel may dissolve at temperatures about 25° C. to 75° C., or the mixture may need to be heated to elevated temperatures.

b) Combining the Solution of Step a) with an Ether.

The solution of docetaxel obtained in step a) is combined with an ether solvent such as diethyl ether, diisopropyl ether, methyl tert-butyl ether, and the like, or any combinations thereof, for precipitation.

Suitable temperatures for solid precipitation may range from about 0° C. to about 50° C., or from about 25° C. to about 35° C.

c) Isolating Crystalline Docetaxel.

The obtained precipitate may be separated using conventional techniques known in the art and the solid may optionally be washed with a suitable ether solvent, and the isolated solid may optionally be further dried or the wet solid may be used directly for the next level of purification.

In embodiments, purification of docetaxel is carried out by providing a solution of docetaxel in a solvent mixture of dichloromethane and acetonitrile, and precipitating a solid by combining the solution with diisopropyl ether.

II. Purification Using a Ketone, Halogenated Hydrocarbon, Ester, or any Mixtures Thereof, and Toluene.

a) Combining Docetaxel with a Ketone or Halogenated Hydrocarbon or Ester Solvent or Mixtures Thereof.

Docetaxel is combined with: a ketone solvent such as acetone, methyl isobutyl ketone, methyl ethyl ketone, and the like: a halogenated hydrocarbon solvent such as dichloromethane, chloroform, and the like; an ester solvent such as ethyl acetate, n-propyl acetate, n-butyl acetate and the like; and any mixtures thereof to provide a solution or suspension. Depending on the solvent and its quantity used, docetaxel may dissolve at temperatures about 25° C. or higher.

b) Adding Toluene to the Solution or Suspension Obtained.

The solution or suspension of docetaxel is combined with toluene and the obtained suspension can be stirred for about 30 minutes to about 2 hours, or longer.

The amount of toluene used may range from about 5 mL to about 30 mL, per gram of the starting docetaxel.

c) Isolating Crystalline Docetaxel.

The formed solid may be separated using conventional techniques known in the art and the solid may optionally be washed with toluene or an ether and the isolated solid may optionally be further dried or the wet compound may be progressed directly to the next level of purification.

In embodiments, the purification of docetaxel is carried out by providing a solution of docetaxel in methyl isobutyl ketone, and precipitating the compound by adding toluene.

In embodiments, the purification of docetaxel is carried out by providing a solution of docetaxel in methyl ethyl ketone, and precipitating the compound by adding toluene.

The above crystallization processes can result in docetaxel having purity greater than or equal to about 98%, as determined using HPLC.

III. Purification Using a Ketone Solvent and an Ether.

a) Dissolving the Docetaxel Obtained in Steps a) in a Ketone Solvent.

A solution of docetaxel can be provided by dissolving docetaxel in a ketone solvent, such as acetone, methyl isobutyl ketone, methyl ethyl ketone, and the like. Solutions of docetaxel can be provided at temperatures about 25° C. to about 45° C., or higher.

b) Adding Ether to the Solution.

Suitable ethers that can used in the process of the present invention for precipitation include, but are not limited to, diethyl ether, diisopropyl ether, tetrahydrofuran (THF), 1,4-dioxane, dimethoxyethane, methyl tert-butyl ether, and the like.

In embodiment, diisopropyl ether is used to precipitate docetaxel. The obtained suspension can be stirred for about 30 minutes to about 2 hours, or longer, to increase the extent of solid formation.

c) Isolating Crystalline Docetaxel.

The obtained solid precipitate may be isolated using conventional techniques known in the art and the solid may optionally be washed with a suitable solvent such as n-hexane, n-heptane, or cyclohexane. The wet solid may optionally be further dried

The above processes of the present invention result in substantially pure docetaxel, having purity greater than or equal to about 99%, or greater than or equal to about 99.5%, and each of the impurities, viz. Impurity I, Impurity II, III, Impurity IV, Impurity V, Impurity VI, Impurity VII and Impurity VIII, Impurity IX (2′,3′-epidocetaxel), Impurity X (2′-epidocetaxel), Impurity XI (10-oxo-docetaxel), Impurity XII (7-epi-docetaxel), Impurity XIII (10-oxo-7-epidocetaxel), may be present at less than about 0.15% as determined using HPLC.

Impurity compounds not described previously are shown in Table 3.

TABLE 3

| Impurity | Structure |
|---|---|
| Impurity IX | 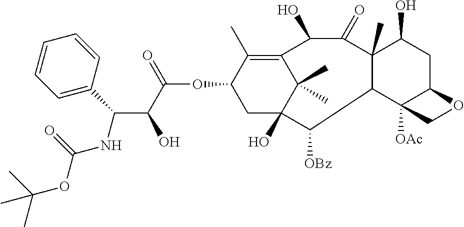 |
| Impurity X | 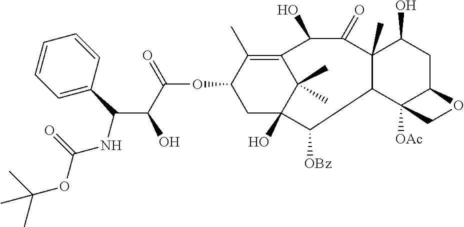 |
| Impurity XI | 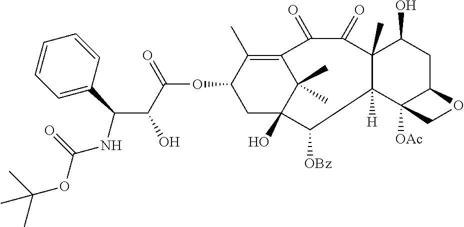 |
| Impurity XII | 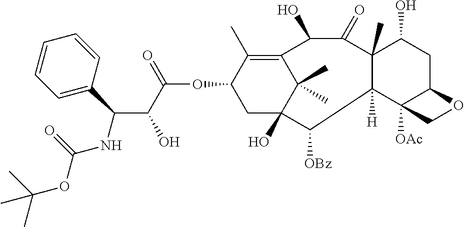 |
| Impurity XIII | 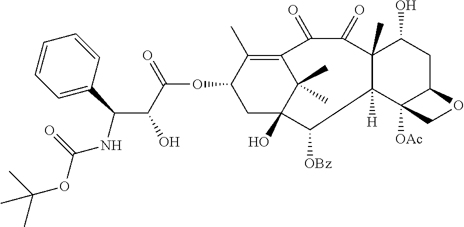 |

High performance liquid chromatography analysis parameters for determining compound purities and impurity amounts are described below.

HPLC Analysis of the Intermediate Compound of Formula II.

Column: ACE 3 C18 150×4.6 mm, 3 μm.

Buffer: 1.36 g of potassium dihydrogen phosphate dissolved in 1000 mL of purified water, pH adjusted to 3±0.05 using dilute phosphoric acid, and filtered through a 0.45 μm porosity membrane.

Mobile Phase (Eluent) A: buffer.

Mobile Phase (Eluent) B: filtered and degassed mixture of acetonitrile and water (90:10 v/v).

Flow rate: 1.0 mL/minute.

Wavelength of detection: 230 nm by UV.

Column temperature: 32±2° C.

Injection volume: 20 μL.

Diluent: Acetonitrile.

Gradient program (values in volume %):

| Minutes | Mobile Phase A | Mobile Phase B |
|---|---|---|
| 0.01 | 60 | 40 |
| 10 | 50 | 50 |
| 25 | 30 | 70 |
| 35 | 10 | 90 |
| 40 | 5 | 95 |
| 70 | 5 | 95 |
| 71 | 60 | 40 |
| 80 | 60 | 40 |

Under these conditions, the compound of formula II may be analyzed for the content of Impurity IV and Impurity VIII, with representative relative retention times as given in Table 4 (compound of Formula 11=1).

TABLE 4

| Impurity | RRT |
|---|---|
| Impurity IV | ~0.95 |
| 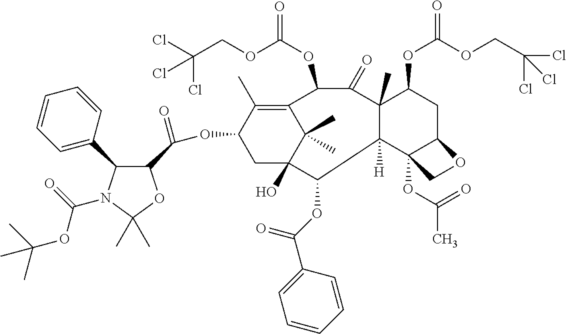 | |
| (2′-epi-isomer of formula II) | |
| Impurity VIII | ~0.9 |
| 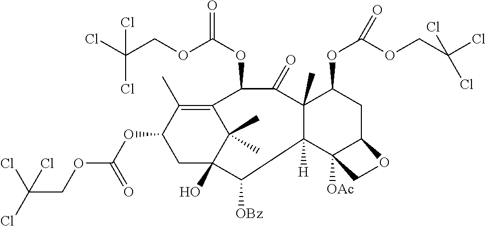 | |
| 7,10,13-tritroc-10-deacetylbaccatin-III | |

HPLC Analysis of the Intermediate Compound of Formula IV.

Column: ACE 3 C18-HL 250×4.6 mm, 5 μm.

Buffer: 1.36 g of potassium dihydrogen phosphate dissolved in 1000 mL of purified water, pH adjusted to 3±0.05 using dilute phosphoric acid, and filtered through a 0.45 μm porosity membrane.

Mobile Phase (Eluent) A: buffer.

Mobile Phase (Eluent) B: filtered and degassed mixture of acetonitrile and water (95:5 v/v).

Flow rate: 1.0 mL/minute.

Wavelength of detection: 230 nm by UV.

Column Temperature: 32±2° C.

Injection volume: 20 μL.

Diluent: acetonitrile.

Gradient program (values in volume %):

| Minutes | Mobile Phase A | Mobile Phase B |
|---|---|---|
| 0.01 | 60 | 40 |
| 50 | 55 | 45 |
| 60 | 30 | 70 |
| 80 | 20 | 80 |
| 100 | 10 | 90 |
| 120 | 10 | 90 |
| 121 | 60 | 40 |
| 130 | 60 | 40 |

HPLC Analysis of Docetaxel.

Column: ACE 3 C18-HL 250×4.6 mm, 5 μm.

Buffer: 1.36 g of potassium dihydrogen phosphate dissolved in 1000 mL of purified water, pH adjusted to 3±0.05 using dilute phosphoric acid, and filtered through a 0.45 μm porosity membrane.

Eluent A: buffer.

Eluent B: filtered acetonitrile.

Flow rate: 1.0 mL/minute.

Wavelength of detection: 230 nm by UV.

Column Temperature: 32±2° C.

Injection volume: 20 μL.

Diluent: acetonitrile.

Gradient program: (values in volume %):

| Minutes | Mobile Phase A | Mobile Phase B |
|---|---|---|
| 0.01 | 60 | 40 |
| 50 | 55 | 45 |
| 60 | 30 | 70 |
| 70 | 5 | 95 |
| 80 | 5 | 95 |
| 81 | 60 | 40 |
| 90 | 60 | 40 |

Under these conditions, docetaxel may be analyzed for the content of impurities with representative relative retention times as given in Table 5 (docetaxel=1).

TABLE 5

| Impurity | RRT |
|---|---|
| 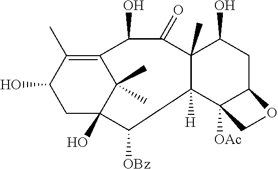 | ~0.12 |
| 10-deacetyl baccatin III | |
| Impurity I | ~0.87 |
| (Oxetane ring opened impurity) | |
| Impurity II | ~0.75 |
| (Oxetane ring opened impurity) | |
| 2′,3′-epi docetaxel | ~0.93 |
| 2′-Epi docetaxel | ~0.95 |
| 10-oxo docetaxel | ~1.45 |
| 7-epi docetaxel | ~1.64 |
| 10-oxo,7-epi docetaxel | ~1.79 |
| Formula IV | ~2.04 |

Some variations in the relative retention times may be expected, based on the specific HPLC instrument employed, system conditions, the analyst, and the sample preparation technique.

Certain specific aspects and embodiments of the present application will be explained in more detail with reference to the following examples, which are provided for purposes of illustration only and should not be construed as limiting the scope of the invention in any manner.

### Example 1

### Preparation of the Intermediate Compound of Formula II, Substantially Free from Isomeric Impurity IV

54 g of (4S,5R)-3-tert-butoxycarbonyl-2,2-dimethyl-4-phenyl-5-oxazolidine carboxylic acid and 1000 mL of dichloromethane are charged into a round bottom flask and stirred for dissolution. 100 g of 4-acetoxy-2α-benzoyloxy-5β,20-epoxy-1β,13α-dihydroxy-9-oxo-7β,10β-bis(2,2,2-trichloroethoxy carbonyloxy)-11-taxene, 13.6 g of dimethylamino pyridine (DMAP), and 46.1 g of dicylohexyldicarbamide (DCC) are added to the solution and the mixture is stirred for 1 hour at temperatures between 25° C. and 35° C. 11.5 g of DCC is added and stirred for 30 minutes. The mixture is filtered and washed with 200 mL of dichloromethane. The filtrate is washed with 500 mL of aqueous saturated NaHCO₃ solution, then with 1000 mL of water. The organic layer is concentrated to 300 mL at 45° C. under vacuum. The organic layer is filtered to remove a dicyclohexylurea by-product and then is washed with 100 mL of dichloromethane. The filtrate is added slowly to 1600 mL of n-heptane in a round bottom flask at 25-30° C. over 40-60 minutes, and the mass is stirred for 1 hour. The obtained suspension is filtered and the solid is washed with 200 mL of n-heptane. The solid is dried at 50° C. for 4 hours under high vacuum, to afford 122.6 g of the title compound.

Yield: 93.7%.

Purity by HPLC: 95.12% formula II.

Impurities by HPLC: Impurity IV: 0.45%; 2.59% other impurity.

Purification Process:

122 g of the compound of formula II (Purity: 95.12%) and 488 mL of dichloromethane are charged into a round bottom flask at 26° C. and stirred for dissolution. The solution is slowly added to 2440 mL of n-heptane in a round bottom flask and stirred for 1 hour at 25-30° C. The suspension is filtered and the solid is washed with 200 mL of n-heptane. The solid is dried at 50° C. for 4 hours under high vacuum, to afford 99 g of the compound of Formula II.

Purity by HPLC: 98.47% formula II.

Impurities by HPLC: Impurity IV: 0.11%; Impurity VIII: 0.94%.

### Example 2

### Preparation of the Intermediate Compound of Formula IV

1250 mL of formic acid and 50 g of the compound of formula II are charged into a round bottom flask and stirred for 45-60 minutes at 25-30° C. 1000 mL of ethyl acetate is added, followed by 2000 mL of water and 1250 mL of brine solution. The mixture is stirred for 10 minutes and the organic layer is separated. The aqueous layer is extracted with 1000 mL of ethyl acetate. The organic layers are combined, washed with 3×1000 mL of 7% sodium bicarbonate solution, and concentrated to about 500 mL. 500 mL of water is added and the pH is adjusted to 7.5 to 8.5 using sodium bicarbonate (about 8.5 g). 11.5 mL of di-tert-butyl dicarbonate is added and stirred for 3 hours at 25-30° C. The organic layer is separated and concentrated to about 100-150 mL at 48° C. under vacuum. The organic layer is added to 2250 mL of petroleum ether in a round bottom flask at 27° C., and stirred for 1 hour. The precipitate obtained is filtered, washed with 128 mL of petroleum ether, and suction dried for 1 hour.

Yield: 47.5 g.

Purity by HPLC: 95.68%.

Impurities by HPLC: Unidentified (at ˜0.84 RRT): 0.36%; Impurity I: 0.22%;

Impurity II: 0.26%; Unidentified (at ˜0.99 RRT): 0.11%; Unidentified (at ˜1.01 RRT): 0.54%; Impurity VIII: 1.09%; and Impurity III: 0.99%.

### Example 3

### Purification of the Intermediate Compound of Formula IV

58 g of the compound of formula IV (purity: 88.53%) and 58 mL of acetonitrile are placed into a round bottom flask at 27° C. and stirred for 10 minutes. 1160 mL of diisopropyl ether is added to the solution and stirred for 2 hours. The suspension obtained is filtered and the solid is washed with 116 mL of diisopropyl ether. The solid is dried under high vacuum at 45-50° C. for 2 hours.

Yield: 44.0 g.

Purity by HPLC: 98.68%.

### Example 4

### Preparation of Docetaxel

30 g of the compound of formula IV and 300 mL of acetone are charged into a round bottom flask and stirred at 27° C. for dissolution. 13.5 g of zinc dust and 30 mL of acetic acid are added and stirred for 30 minutes at 25-30° C., until completion of the reaction. The mass is filtered through diatomaceous earth and the bed is washed with 40 mL of acetone. The filtrate is charged into 3000 mL of water. The mixture is stirred for 1 hour at 25 to 35° C. and then filtered. The obtained wet cake is washed with 60 mL of water, suction dried for 30 minutes, and then dried at 45-50° C. under high vacuum for 4 hours.

Yield: 20.1 g (96.17%).

Purity by HPLC: 92.14%.

Impurities by HPLC: 10-oxo DCT: 0.33%; 7-epi DCT: 0.40%; 10-oxo-7-epi DCT: 0.19%; Impurity VI: 1.60%; and Impurity V: 2.10%.

Purification using column chromatography.

A column is packed with silica gel (230-400 mesh, 487.5 g) in 20% ethyl acetate in petroleum ether (1000 mL). Docetaxel (19.5 g, 92.14% purity) is dissolved in dichloromethane (39 mL) at 40-45° C., cooled to 25-35° C., and charged to the column. The column is eluted with a mixture of ethyl acetate and petroleum ether (1000 mL of 20% ethyl acetate in petroleum ether, 1000 mL of 40% ethyl acetate in petroleum ether, and 8 L of 45% ethyl acetate in petroleum ether). The desired pure fractions are collected by monitoring with TLC and pooled to give a purified fraction. The purified fraction is concentrated at 45-50° C. under vacuum to a volume of 55-65 mL, then cooled to 25-30° C. and combined with petroleum ether (390 mL). The mixture is stirred for 30 minutes at 25-30° C. and filtered under vacuum. The wet solid is washed with petroleum ether (40 mL) and dried under high vacuum to give purified docetaxel.

Yield: 15.1 g (77%).

Purity by HPLC: 99.48%.

### Example 5

### Preparation of Substantially Pure Docetaxel

10 g of the compound of formula IV and 50 mL of acetone are charged into a round bottom flask and stirred for 10 minutes at 27° C. for dissolution. 10 mL of acetic acid and 4.51 g of zinc dust are added. The temperature of the mass is raised to 40-45° C. with stirring for 30 minutes. The mass is filtered through diatomaceous earth and the bed is washed with 20 mL of acetone. The filtrate is distilled to 25 mL under vacuum at 48° C. A column is packed with 250 g of 230-240 mesh silica gel in 600 mL of petroleum ether. The concentrated filtrate is loaded into the column and the column is eluted with 1000 mL of petroleum ether and mixtures of ethyl acetate in petroleum ether (600 mL of 20% ethyl acetate in petroleum ether, 600 mL of 40% ethyl acetate in petroleum ether, and 4000 mL of 45% ethyl acetate in petroleum ether). The desired pure fractions are collected, combined, and distilled under vacuum at 48° C. until 30 mL remains. 50 mL of dichloromethane is added to the residue and distilled at 47° C. under vacuum until 30 mL remains. 450 mL of petroleum ether is added to the residue and stirred for 60 minutes. The suspension is filtered, and the solid is washed with 20 mL of petroleum ether and dried at 49° C. under high vacuum for 4 hours, to give 5.3 g of purified docetaxel.

Yield: 75.7%.

Purity by HPLC: 99.2%.

### Example 6

### Preparation of Pure Docetaxel

10 g of the compound of formula IV and 50 mL of acetone are charged into a round bottom flask and stirred for 10 minutes at 27° C. for dissolution. 10 mL of acetic acid and 4.51 g of zinc dust are added and stirred for 30 minutes. The mass is filtered through diatomaceous earth, the bed is washed with 20 mL of acetone, and portions of the filtrate are further treated separately.

### Part I Treatment:

A column is packed with 125 g of 230-240 mesh silica gel in 300 mL of petroleum ether and the column is washed with petroleum ether two to three times for close packing. A portion of the filtrate from above (25 mL) is loaded into the column and eluted first with 300 mL of petroleum ether, and then with mixtures of ethyl acetate and petroleum ether (300 mL of 20% ethyl acetate in petroleum ether, 300 mL of 40% ethyl acetate in petroleum ether, and 2000 mL of 45% ethyl acetate in petroleum ether). The product-containing fractions are collected and distilled under vacuum at 48° C. to a volume of 10-20 mL. The distillation residue is added to 45 mL of petroleum ether over 15 minutes and the suspension is stirred for 30 minutes. The suspension is filtered and the solid is washed with 10 mL of petroleum ether and suction dried for 30 minutes to give 2.2 g of purified docetaxel.

Yield: 63.2%.

Purity by HPLC: 98.66%.

Part II Treatment:

A portion of the filtrate obtained above (35 mL, corresponding to 5 g of the compound of Formula IV) is added to 500 mL of water and stirred for 1 hour. The solid is filtered and washed with 10 mL of water. The solid is suction dried for 1 hour to give 3 g of docetaxel. A column is packed with 75 g of 230-400 mesh silica gel in 200 mL of petroleum ether. The docetaxel (3 g) is dissolved in 24 mL of dichloromethane at 40° C. and then loaded into the column. The column is eluted with mixtures of ethyl acetate and petroleum ether (250 mL of 20% ethyl acetate in petroleum ether, 500 mL of 40% ethyl acetate in petroleum ether, and 1600 mL of 45% ethyl acetate in petroleum ether). Product-containing fractions are collected and concentrated to less than 15 mL under vacuum at 48° C. 45 mL of petroleum ether is added to the residue and stirred for 15 to 30 minutes at 27° C. The suspension is filtered and the solid is suction dried for 30 minutes to give 2.16 g of purified docetaxel.

Yield: 72%.

Purity by HPLC: 99.11%.

### Example 7

### Preparation of Docetaxel

6 g of the compound of formula IV and 120 mL of acetone are charged into a round bottom flask and stirred for 10 minutes at 27° C. for dissolution. 6 mL of acetic acid and 5.4 g of zinc dust are added and stirred for 30 minutes at 40-50° C. The mass is filtered through diatomaceous earth, the bed is washed with 12 mL of acetone, and the filtrate is concentrated below 25° C. under high vacuum, to obtain 9-15 mL of concentrate.

Purification of Docetaxel Using Column Chromatography.

A column is packed with 105 g of 230-240 mesh silica gel in 300 mL of petroleum ether and the column is washed with petroleum ether two to three times for close packing. The above obtained concentrate (9-15 mL) is loaded into the column and eluted first with 600 mL of petroleum ether and then with mixtures of ethyl acetate and petroleum ether (360 mL of 20% ethyl acetate in petroleum ether, 360 mL of 40% ethyl acetate in petroleum ether, and 6000 mL of 45% ethyl acetate in petroleum ether). The desired pure fractions are collected, combined, and distilled under vacuum at 48° C. until 18 mL remains. 30 mL of dichloromethane is added and distilled at 47° C. under vacuum, until 18 mL remains. 270 mL of petroleum ether is added and stirred for 60 minutes. The suspension is filtered, and the solid is washed with 12 mL of petroleum ether and dried at 55-60° C. under high vacuum for 10 hours, to give 3.05 g of purified docetaxel.

Purity by HPLC: >99.0%.

### Example 8

### Preparation of Docetaxel

6 g of the compound of formula IV and 120 mL of acetone are charged into a round bottom flask and stirred for 10 minutes at 27° C. for dissolution. 6 mL of acetic acid and 8.1 g of zinc dust are added and stirred for 30 minutes at 40-50° C. The mass is filtered through diatomaceous earth, the bed is washed with 12 mL of acetone, and the filtrate is concentrated at below 25° C. using high vacuum, to obtain 9-15 mL of concentrate.

Purification of Docetaxel Using Column Chromatography.

A column is packed with 150 g of 230-240 mesh silica gel in 300 mL of petroleum ether and the column is washed with petroleum ether two to three times for close packing. The above obtained concentrate (9-15 mL) is loaded into the column and eluted first with 600 mL of petroleum ether and then with mixtures of ethyl acetate and petroleum ether (360 mL of 20% ethyl acetate in petroleum ether, 360 mL of 40% ethyl acetate in petroleum ether, and 6000 mL of 45% ethyl acetate in petroleum ether). The desired pure fractions are collected, combined, and distilled under vacuum at 48° C., until 18 mL remains. 30 mL of dichloromethane is added and distilled at 47° C. under vacuum, until 18 mL remains. 270 mL of petroleum ether is added and stirred for 60 minutes. The suspension is filtered, and the solid is washed with 12 mL of petroleum ether and dried at 55-60° C. under high vacuum for 10 hours, to give 3.15 g of purified docetaxel.

Purity by HPLC: >99.0%.

### Example 9

### Preparation of Docetaxel

75 g of the compound of formula IV and 1500 mL of acetone are charged in a round bottom flask under a nitrogen atmosphere and stirred. 75 mL of acetic acid and 67.6 g of zinc dust are added and stirred for 30 minutes at 40-45° C. The mass is filtered through diatomaceous earth, the bed is washed with 225 mL of acetone, and the filtrate is concentrated below 25° C. under high vacuum, to obtain about 600 mL of concentrate. The concentrate is added to 7500 mL of water, stirred at 25-30° C. for about 1 hour, filtered, washed with 375 mL water and suction dried under vacuum for 1 hour.

1875 mL of dichloromethane and the obtained solid are charged into a round bottom flask at 25-30° C. and stirred for dissolution. The mass is washed with 750 mL of saturated sodium chloride solution, and the organic layer is separated and portions are separately further treated.

Part I Treatment:

A portion of the organic layer (˜1250 mL) is distilled to obtain 150 mL of concentrate and then mixed with 600 mL of n-heptane. The mass is distilled to obtain 250 mL of concentrate, which is combined with 500 mL of n-heptane. The mass is distilled to obtain 600 mL of concentrate, which is stirred for 1 hour at 25-30° C. and filtered. The solid is washed with 100 mL of n-heptane, suction dried for 15 minutes, and dried under high vacuum at 45-50° C. for about 4 hours.

Yield: 33.3 g.

Purity by HPLC: 94.36%.

1 g of the solid obtained and 5 mL of methyl isobutyl ketone are placed into a round bottom flask and stirred for 10 minutes at 25-30° C. 10 mL of toluene is added and stirred for 1 hour. The solid is filtered, washed with 2 mL of toluene and suction dried under vacuum for 60 minutes.

Yield: 0.78 g.

Purity by HPLC: 98.36%.

Impurities by HPLC: Impurity I: 0.06%; Impurity II: not detected; 2′,3′-epi docetaxel: 0.02%; 2′-epi docetaxel: 0.03%; 10-oxo docetaxel: 0.01%; 10-oxo-7-epi docetaxel: not detected; Impurity V: not detected; Impurity VI: not detected.

Part II Treatment:

A portion of the organic layer (˜625 mL) is distilled to obtain 75 mL of concentrate and then mixed with 125 mL of acetonitrile. The mass is distilled to obtain 50 mL of concentrate, which is mixed with 375 mL of diisopropyl ether and stirred for 1 hour at 25-30° C. The solid is filtered, washed with 50 mL of n-heptane, suction dried under vacuum for 15 minutes, and dried under high vacuum at 45-50° C. for about 4 hours.

Yield: 15.7 g.

Purity by HPLC: 97.58%.

Impurities by HPLC: 10-deacetylbaccatin III: not detected; Impurity I: 0.05%; Impurity II: not detected; 2′,3′-epi docetaxel: not detected; 2′-epi docetaxel: 0.19%; 10-oxo docetaxel: 0.09%; 7-epi docetaxel: 0.77%; formula IV: not detected; 10-oxo-7-epi docetaxel: not detected; Impurity V: 0.83%; Impurity VI: 0.39%.

### Example 10

### Purification of Docetaxel

1 g of docetaxel (purity: 94.36%, obtained in Example 9, part I) and 5 mL of methyl ethyl ketone are placed into a round bottom flask at 27° C. and stirred for 10 minutes. 10 mL of toluene is added and stirred for 1 hour. The suspension is filtered and the solid is washed with 3 mL of toluene and suction dried under vacuum for 60 minutes.

Yield: 0.82 g (wet).

Purity by HPLC: 98.46%.

Impurities by HPLC: Impurity I: 0.07%; Impurity II: not detected; Impurity V: not detected; Impurity VI: not detected; 2′,3′-epi docetaxel: 0.09%; 2′-epi docetaxel: not detected; 10-oxo docetaxel: 0.16%; 7-epi-docetaxel: 0.21%; 10-oxo-7-epi docetaxel: 0.06%.

0.5 g of the solid obtained and 10 mL of acetone are placed into a round bottom flask and stirred for 10 minutes at 27° C. 30 mL of diisopropyl ether is added slowly and stirred for 90 minutes. The solid is filtered, washed with 2 mL of diisopropyl ether, and suction dried under vacuum for 60 minutes.

Yield: 0.39 g.

Purity by HPLC: 99.3%.

Impurities by HPLC: Impurity I: 0.03%; Impurity II: not detected; Impurity V: not detected; Impurity VI: not detected; 2′,3′-epi docetaxel: 0.02%; 2′-epi docetaxel: 0.03%; 10-oxodocetaxel: 0.1%; 7-epi-docetaxel: 0.04%; 10-oxo-7-epi docetaxel: not detected.

### Example 11

### Purification of Docetaxel

1 g of docetaxel (purity: 97.19%, 7-epi docetaxel: 0.81, 2′-epi docetaxel: 0.26%) and 5 mL of methyl ethyl ketone are placed into a round bottom flask at 27° C. and stirred for 10 minutes. 10 mL of toluene is added and stirred for 1 hour. The suspension is filtered and the solid is washed with 5 mL of diisopropyl ether suction dried for 60 minutes.

Yield: 0.74 g (wet).

Purity by HPLC: 98.77%.

Impurities by HPLC: 7-epi docetaxel: 0.2%; 2′-epi docetaxel: 0.04%.

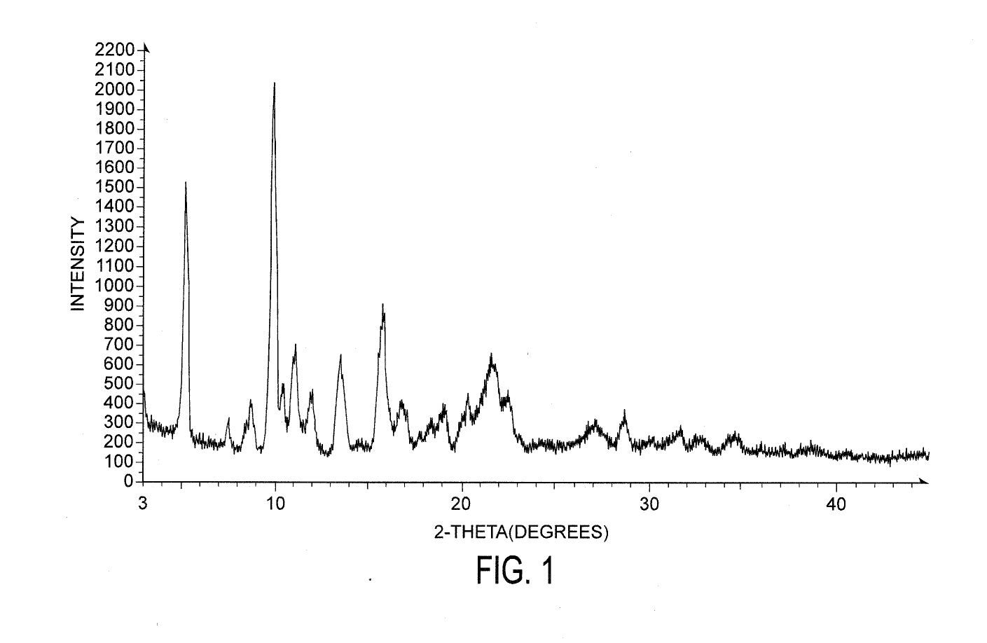

XRPD pattern (using copper Kα radiation): as shown in FIG. 1.

1. A process for preparing docetaxel, comprising:
a) reacting 4-acetoxy-2α-benzoyloxy-5β,20-epoxy-1-hydroxy-9-oxo-7β,10β-bis[(2,2,2-tri-chloroethoxy)carbonyloxy]-11-taxen-13α-yl(4S,5R)-3-tert-butoxycarbonyl-2,2-dimethyl-4-phenyl-5-oxazolidinecarboxylate of formula II, 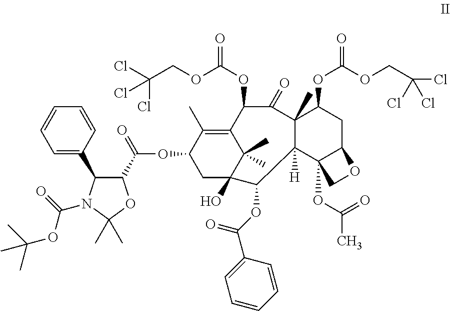
with formic acid at about 20 to 40° C. for no more than about 2 hours, to obtain 4-acetoxy-2α-benzoyloxy-5β,20-epoxy-1-hydroxy-9-oxo-7β,10β-bis[(2,2,2-trichloro ethoxy)carbonyloxy]-11-taxen-13α-yl (2R,3S)-3-amino-2-hydroxy-3-phenylpropionate of formula III; 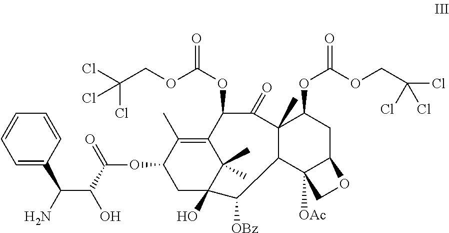
b) reacting the compound of formula III with 1 to 1.5 molar equivalents of di-tert-butyl dicarbonate in a bi-phasic medium to obtain 4-acetoxy-2α-benzoyloxy-5β,20-epoxy-1-hydroxy-9-oxo-7β,10β-bis[(2,2,2-trichloroethoxy)carbonyloxy]-1′-taxen-13α-yl(2R,3S)-3-tert-butoxycarbonylamino-2-hydroxy-3-phenylpropionate of formula IV; and 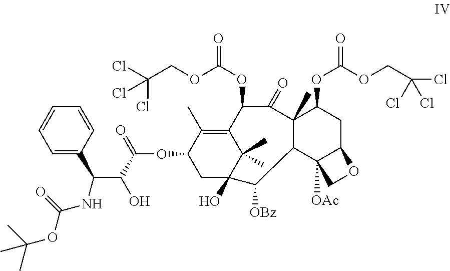
c) converting the compound of formula IV to docetaxel.

2. The process of claim 1, wherein an amount of formic acid used is about 5-30 mL, per gram of the compound of formula II.

3. The process of claim 1, wherein a bi-phasic medium comprises water and an ester solvent.

4. The process of claim 1, wherein b) is conducted at pH values about 7-9.

5. A process for preparing docetaxel, comprising reacting a compound of formula IV, 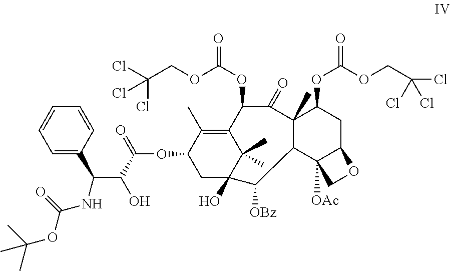
with zinc in the presence of an organic acid and a ketone solvent.

6. The process of claim 5, wherein an organic acid comprises acetic acid, propionic acid, butanoic acid, or oxalic acid.

7. The process of claim 5, wherein a ketone comprises acetone, methyl isobutyl ketone, or ethyl methyl ketone.

8. The process of claim 5, wherein an amount of acetic acid used is about 1 to 3 mL, per gram of the compound of formula IV.

9. The process of claim 5, further comprising recovering docetaxel by concentrating a reaction mass, or by combining a reaction mass with an anti-solvent comprising water, an alkane, or an ether.

10. A process for purifying a compound of formula IV, comprising: 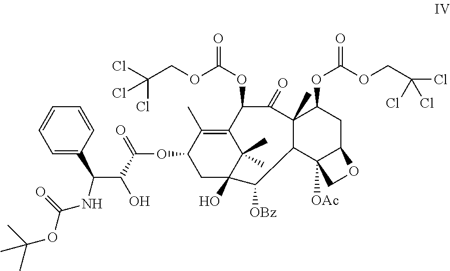
(a) providing a solution of a compound of formula IV in an organic solvent;
(b) combining the solution of (a) with an anti-solvent;
(c) isolating the compound of formula IV; and
(d) optionally repeating (a)-(c) to obtain a desired purity.

11. The process of claim 10, wherein an organic solvent comprises an ester, a nitrile, or a halogenated alkane.

12. The process of claim 10, wherein an organic solvent comprises ethyl acetate, butyl acetate, isopropyl acetate, acetonitrile, propionitrile, dichloromethane, or chloroform.

13. The process of claim 10, wherein an anti-solvent comprises an ether.

14. A process for preparing substantially pure docetaxel, comprising any of I, II, or III, or any combination thereof:
I. Purification in a solvent comprising a nitrile and ether:
a) dissolving the docetaxel in a solvent comprising a nitrile;
b) combining the solution of step a) with an ether; and
c) isolating crystalline docetaxel;
II. Purification using a ketone, halogenated hydrocarbon, ester, or mixtures thereof, and toluene:
a) combining docetaxel with a ketone, halogenated hydrocarbon, ester, or mixtures thereof;
b) adding toluene to the solution or suspension; and
c) isolating crystalline docetaxel;
III. Purification using a ketone solvent and an ether:
a) dissolving docetaxel in a ketone solvent;
b) adding ether to the solution; and
c) isolating crystalline docetaxel.

15. The process of claim 14, wherein I, II, and III are performed sequentially.

16. The process of claim 14, wherein a ketone comprises acetone, methyl isobutyl ketone or methyl ethyl ketone.

17. The process of claim 14, wherein a halogenated hydrocarbon comprises dichloromethane or chloroform.

18. The process of claim 14, wherein an ester solvent comprises ethyl acetate, n-butyl acetate, or n-propyl acetate.

19. The process of claim 14, wherein an ether comprises diethyl ether, diisopropyl ether or methyl tert-butyl ether.

20. Docetaxel, containing less than about 0.15 percent by weight of each of impurities V and VI. [chemical structure]
